(19) United States
(12) Patent Application Publication
(10) Pub. No.: US 2003/0187073 A1
(43) Pub. Date: Oct. 2, 2003

(54) Disinfectant agent

(76) Inventors: Florian Lichtenberg, Grenzach-Wyhlen (DE); Michael Lützeler, Grenzach-Wyhlen (DE); Volker Ranft, Murg (DE)

Correspondence Address: Fisher Christen & Sabol, Suite 1108, 1725 K Street N W, Washington, DC, 20006, US

(21) Appl. No.: 10/381,012

(22) PCT Filed: Sep. 18, 2001
(86) PCT No.: PCT/EP01/10755

(30) Foreign Application Priority Data
Sep. 20, 2000 (EP) 001205863

(51) Int. Cl. A61K 31/155; A61K 31/14; A61L 9/00

(52) U.S. Cl. 514/634; 514/642; 422/28

(57) ABSTRACT

The invention relates to compositions of a disinfectant agent containing a) at least one amine and/or quaternary ammonium salt of general formula (Ia) or (Ib), wherein R¹ C₆₋₁₈ represents alkyl, R² represents benzyl or C₆₋₁₈ alkyl; R³ C₁₋₁₈ represents alkyl or —[(CH₂)₂—O)ₙR₆ whereby n=1-20; R⁴ and R⁵ represent independently from each other C₁₋₄ alkyl; R⁶ represents hydrogen or optionally substituted phenyl and A represents a monovalent anion or an equivalent multivalent anion of an inorganic or organic acid; and b) at least one guanidinium salt of formula (II), wherein X represents a monovalent anion or an equivalent multivalent anion of an organic or inorganic acid, in a mass ratio (I):(II) of 20:1-1:20. The inventive compositions are characterised in that they have a good bacterial and equally good fungicidal efficacy when used in low concentrations and can also be used as disinfectant agents as well as preservation agents.

The invention relates to synergistic disinfectant compositions based on amines and/or quaternary ammonium salts.

Numerous disinfectant and preservative compositions based on amines and/or quaternary ammonium salts are known. However, in general, in particular at relatively high dilution, these exhibit an unsatisfactory activity towards fungi, for example Aspergillus niger.

It was therefore an object of the present invention to provide disinfectant compositions based on amines and/or quaternary ammonium salts which exhibit good activity towards fungi even at high dilution.

This object is achieved according to the invention by the disinfectant composition according to claim 1.

It has surprisingly been found that amines and/or quaternary ammonium salts of the general formula

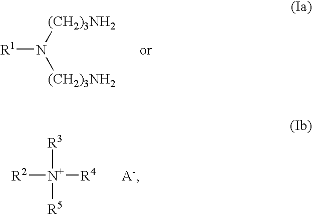

where R¹ is C₆₋₁₉-alkyl

R² is benzyl or C₆₋₁₈-alkyl

R³ is C₁₋₁₈-alkyl or —[(CH₂)₂—O]ₙR⁶ where n=1-20

R⁴ and R⁵ independently of one another are C₁₋₄-alkyl

R⁶ is hydrogen or unsubstituted or substituted phenyl and A⁻ is a monovalent anion or one equivalent of a polyvalent anion of an inorganic or organic acid;

by addition of at least one guanidinium salt of the formula

poly(oxyethyl)ammonium salt or a mixture of two or more of these compounds.

The anion X⁻ of the guanidinium salt is preferably chloride, bromide, rhodanide, nitrate, hydrogen sulfate, sulfate, dihydrogen phosphate, monohydrogen phosphate, phosphate, carbonate or acetate. It is also possible to use a mixture of two or more of such salts.

Particularly good results have been achieved using guanidinium carbonate [(CH₆N₃)₂CO₃].

The mass ratio of amine (Ia) or quaternary ammonium salt (Ib) to guanidinium salt (II) is preferably in the range from 1:5 to 5:1.

The inventive disinfectant compositions preferably comprise water as solvent, if appropriate in combination with an organic solvent.

Preferably, the inventive disinfectant compositions further comprise one or more aids selected from the group consisting of organic solvents, surfactants, complexing agents, fragrances and colorants.

A preferred field of application of the inventive disinfectant compositions is surface disinfection and instrument disinfection.

Further preferred fields of application are laundry disinfection and hand disinfection.

The inventive disinfectant compositions are also suitable for use in chemical toilets, for example on board aircraft and vehicles.

A further preferred field of use is the preservation of industrial liquids, for example water circulation in paper manufacturing, cooling water, belt lubricants for conveyor belts, or cutting fluids in metal machining.

An application which is likewise preferred is finally the use as preservative for construction materials which are organic or susceptible to biological attack, for example wood.

The examples below illustrate the implementation of the invention, and should not be taken to be a restriction to the embodiments described. All quantities given, where not otherwise specified, are in % by mass. The test microorganism used in each case was Aspergillus niger ATCC 16404.

### EXAMPLE 1

A disinfecting, cleaner formulation (concentrate) was prepared from:

5.0% didecyldimethylammonium chloride (50% strength solution)

2.0% N,N-bis(3-aminopropyl)dodecylamine

5.0% guanidinium carbonate

5.0% Genapol® T250 (tallow fatty alcohol polyglycol ether, 25 mol of ethylene oxide)

0.5% sodium metasilicate

0.25% sodium carbonate

3.0% ethylene-diaminetetraacetic acid tetrasodium salt (40% strength solution) water to 100%

The effectiveness was determined using a dilution (1 part of concentrate, 99 parts of water) at 20° C. and with a contact time of 15 min. Test microorganism was. The logarithm to base ten of the reduction in microorganism count was 4.0.

### COMPARATIVE EXAMPLE 1

The procedure of Example 1 was followed, but with the difference that the guanidinium carbonate was replaced by the same amount of water. Under the same test conditions, the formulation was virtually inactive.

### EXAMPLE 2

A disinfectant formulation (concentrate) was prepared from:

4.9% N,N-bis(3-aminopropyl)dodecylamine

4.0% guanidinium carbonate

2.0% Genapol® T250 (tallow fatty alcohol polyglycol ether, 25 mol of ethylene oxide)

5.0% Hostapur® SAS 30 (C₁₃₋₁₇ secondary n-alkanesulfonic acid, sodium salt)

2.0% ethylenediaminetetraacetic acid tetrasodium salt (40% strength solution)

0.7% ethylenediaminetetraacatic acid water to 100%

The effectiveness was determined using a dilution (1 part of concentrate, 99 parts of water) at 20° C. and with a contact time of 15 min. The logarithm to base ten of the reduction in microorganism count was 4.1.

### EXAMPLE 3-15

Aqueous solutions were prepared from 0.5% guanidinium salt (II) and 0.25 [lacuna] of amine or quaternary ammonium salt (Ia/Ib) and tested using the method specified in CEN 1275. The results are summarized in Table 1.

TABLE I

| | | | log |
|---|---|---|---|
| Example | | | microbial |
| No. | Amine/ammonium salt | Guanidinium salt | reduction |
| 3 | didecyldimethyl- | carbonate | >4.4 |
| | ammonium chloride | | |
| 4 | didecyldimethyl- | acetate | 3.9 |
| | ammonium chloride | | |
| 5 | didecyldimethyl- | nitrate | 2.3 |
| | ammonium chloride | | |
| 6 | didecyldimethyl- | chloride | 3.0 |
| | ammonium chloride | | |
| 7 | didecyldimethyl- | sulfate | 2.8 |
| | ammonium chloride | | |
| 8 | N,N-bis(3-amino- | carbonate | >4.4 |
| | propyl)dodecylamine | | |
| 9 | N,N-bis(3-amino- | acetate | 4.0 |
| | propyl)dodecylamine | | |
| 10 | N,N-bis(3-amino- | nitrate | 3.3 |
| | propyl)dodecylamine | | |
| 11 | N,N-bis(3-amino- | chloride | 3.5 |
| | propyl)dodecylamine | | |
| 12 | N,N-bis(3-amino- | sulfate | 2.8 |
| | propyl)dodecylamine | | |
| 13 | dimethyldioctyl- | carbonate | >4.4 |
| | ammonium chloride | | |
| 14 | di-C₈₋₁₀-alkyldimethyl- | carbonate | 3.7 |
| | ammionium chloride | | |
| | (60%)/C₁₂₋₁₆-alkyl- | | |
| | benzyldimethylammonium | | |
| | chloride (40%); | | |
| | Bardac ®205-M | | |
| 15 | Benzyldimethyl-2-[2-[4- | carbonate | 2.7 |
| | (1,1,3,3-tetramethyl- | | |
| | butyl)phenoxy]ethoxy]- | | |
| | ethylammonium chloride | | |

For comparison, all compounds listed in Table 1 were tested as individual substances in 0.5% strength solution. None of these compounds exhibited pronounced fungicidal activity (log microbial reduction <2).

1. Disinfectant composition comprising
a) at least one amine and/or quaternary ammonium salt of the general formula 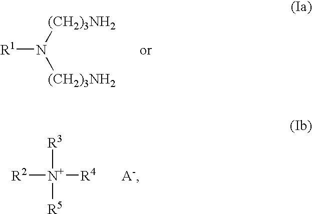
where R¹ is C₆₋₁₈-alkyl
R² is benzyl or C₆₋₁₈-alkyl
R³ is C₁₋₁₈-alkyl or —[(CH₂)₂—O]ₙR⁶ where n=1-20
R⁴ and R⁵ independently of one another are C₁₋₄-alkyl
R⁶ is hydrogen or unsubstituted or substituted phenyl
and A⁻ is a monovalent anion or one equivalent of a polyvalent anion of an inorganic or organic acid; and
b) at least one guanidinium salt of the formula 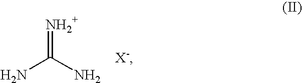
where X⁻ is a monovalent anion or one equivalent of a polyvalent anion of an inorganic or organic acid,
in the mass ratio (I):(II) of 20:1 to 1:20.

2. Disinfectant composition according to claim 1, characterized in that the amine or quaternary ammonium salt is selected from the group consisting of N,N-bis(3-aminopropyl)dodecylamine, N,N-bis(3-aminopropyl)octylamine, didecyldimethylammonium salts, dioctyldimethylammonium salts, octyldecyldimethylammonium salts,
